(19) United States
(12) Patent Application Publication
(10) Pub. No.: US 2014/0329983 A1
(43) Pub. Date: Nov. 6, 2014

(54) Process for the manufacture of vinyl chloride monomer (VCM) and of polyvinyl chloride (PVC)

(71) Applicant: SOLVAY SA, Brussels (BE)

(72) Inventors: Andrea Salto, Bahia Blanca (AR); Maria Martin Carnicero, Brussels (BE); Paul Julius Degraeve, Isieres (BE); Michel Lempereur, Corbais (BE)

(21) Appl. No.: 14/363,096

(22) PCT Filed: Dec. 4, 2012
(86) PCT No.: PCT/EP2012/074344

§ 371 Date: Jun. 5, 2014

(30) Foreign Application Priority Data
Dec. 6, 2011 (EP) 11192035.1

(51) Int. Cl. C07C 17/25; C08F 14/06

(52) U.S. Cl. CPC C07C 17/25; C08F 14/06
USPC 526/344; 570/226

(57) ABSTRACT

Process for the manufacture of vinyl chloride monomer (VCM), comprising step 1): subjecting 1,2-dichloroethane (EDC) to pyrolysis in order to generate a gas mixture comprising VCM, hydrochloric acid (HCl), and EDC; step 2): quenching and/or cooling and/or condensing such gas mixture to a liquid+gas mixture; step 3): subjecting such liquid+gas mixture to a first separation step to remove substantially all the HCl from the liquid+gas mixture so as to leave a stream consisting substantially of VCM and EDC; and step 4): subjecting such VCM+EDC stream to a second separation step so as to get a stream of substantially pure VCM and a stream of unconverted EDC, wherein a heat exchanger is used to heat up the VCM+EDC stream prior to being fed to a distillation column in step 4), such heat exchanger being powered by a stream of hot fluid available in any one of steps 2) to 4) of the process.

The present invention relates to a process for the manufacture of vinyl chloride monomer (VCM) and of polyvinyl chloride (PVC).

For producing VCM, two methods generally are employed: the hydrochlorination of acetylene and the dehydrochlorination of ethylene dichloride (1,2-dichloroethane) or EDC. The latter generally happens by thermal cracking and the EDC used therefore is generally obtained by direct chlorination and/or oxychlorination of ethylene.

As namely explained in “Chemical Process Design: Computer-Aided Case Studies”, Alexandre C. Dimian and Costin Sorin Bildea, Copyright© 2008 WILEY-VCH Verlag GmbH & Co. KGaA, Weinheim, ISBN: 978-3-527-31403-4, Chapter 7 entitled: “Vinyl Chloride Monomer Process”, to date, most of the VCM technologies are based on “balanced” processes.

By this is meant that all intermediates and by-products are recycled in a way that ensures a tight closure of the material balance to only VCM as the final product, starting from ethylene, chlorine and oxygen. The main chemical steps involved are:

1. Direct chlorination of ethylene to 1,2-ethylene dichloride (EDC):

C2H4+Cl2→C2H4Cl2+218kJ/mol

2. Thermal cracking (pyrolysis) of EDC to VCM:

C2H4Cl2→C2H3Cl+HCl−71kJ/mol

3. Recovery of HCl and oxychlorination of ethylene to EDC:

C2H4+2HCl+0.5O2→C2H4Cl2+H2O+238kJ/mol

Hence, an ideal balanced process can be described by the overall equation:

C2H4+0.5Cl2+0.25O2→C2H3Cl+0.5H2O+192.5kJ/mol

As set forth above, the reaction product of the pyrolysis reaction is a gaseous mixture of VCM and HCl and since this reaction is in fact not entirely completed, unconverted EDC is also present in said mixture. This gaseous mixture, which generally is at a high temperature (about 500° C.), is rapidly cooled by quenching and then condensed and the gas+liquid mixture so obtained is then subjected to separation, generally by distillation and generally using at least two steps/columns:

Column 1 (or HCl column): feed: (VCM+HCl+EDC) from cracker/top: HCl (+C2H2)/bottom: (VCM+EDC)
Column 2 (or VCM column): feed: (VCM+EDC) from column 1/top: crude VCM/bottom: unconverted EDC.

This same document sets forth, namely in sub-chapter 7.7, several ways of saving energy in a “balanced” process as described above. One of these ways consists in using the enthalpy of the cracker outlet stream after it has been quenched (in order to prevent decomposition of the VCM produced and to remove coke and other impurities) for ensuring the reboiler duty of column 2. This is possible in the process detailed in that document because of the respective temperatures at the outlet stream (139° C.) and at reboiler (129° C.). However, in many industrial processes, column 2 operates at higher temperature (and pressure) so that this solution cannot be applied.

Patent application CA 1127669 also discloses using the enthalpy of the cracker outlet stream for ensuring the reboiler duty of column 2, but before said stream has been quenched in order to have a sufficient heat (temperature) available for ensuring said duty, considering the fact that said reboiler operates at a temperature of at least 200° C.

The present invention aims at providing a new route for energy saving in a VCM manufacturing process, which also focuses on this VCM column energy consumption, but allows using a stream of lower thermal content.

To this effect, the invention relates to a process for the manufacture of vinyl chloride monomer (VCM), comprising the steps of:

1. subjecting 1,2-dichloroethane (EDC) to pyrolysis in order to generate a gas mixture comprising VCM, HCl and EDC
2. quenching and eventually further cooling and/or condensing said gas mixture to a liquid+gas mixture
3. subjecting said liquid+gas mixture to a first separation step to remove substantially all the HCl there from so as to leave a stream consisting substantially of VCM and EDC
4. subjecting said VCM+EDC stream to a second separation step so as to get a stream of substantially pure VCM and a stream of unconverted EDC, according to which a heat exchanger is used to heat up the VCM+EDC stream prior to being fed to a distillation column in step 4, said heat exchanger being powered by a stream of hot fluid available in any one of steps 2 to 4 of the process but after the quenching of step 2.

In the above, the term [symbol] substantially [symbol] means in fact that there only remains a limited amount of impurities (typically: a few w % or less) in said streams. As to the terms “a stream of hot fluid available in any one of steps 1 to 4 of the process”, they tend to designate any stream of fluid (gas and/or gas+liquid mixture) entering, being inside or leaving any of said steps.

In a first embodiment of the invention, the heat exchanger is powered by at least part of the stream of unconverted EDC obtained in step 4.

In a second embodiment of the invention, the heat exchanger is powered by a stream of hot mixture comprising VCM, HCl and EDC available in step 2 after the quenching.

In step 1 of the process according to the invention, the conditions under which the pyrolysis may be carried out are known to persons skilled in the art. This pyrolysis is advantageously obtained by a reaction in the gaseous phase in a tubular oven. The usual pyrolysis temperatures are between 400 and 600° C. with a preference for the range between 480° C. and 540° C. The residence time is advantageously between 1 and 60 s with a preference for the range from 5 to 25 s. The rate of conversion of the EDC is advantageously limited to 45 to 75% in order to limit the formation of by-products and the fouling of the tubes of the oven. Typically, the gas mixture coming from the pyrolysis is at a pressure from 10 to 25 barg.

In step 2 of the process according to the invention, this gas mixture is first cooled down in a quench device (tower generally) and thereafter, generally partially condensed using at least one condenser but preferably, at least 2 or even more preferably: a train of successive condensers. As used herein, “quench device” is a device for removing some components of the gases (namely coke particles that are generally generated during pyrolysis) there from by putting a sufficient quantity of liquid quench medium (generally a liquid mixture of VCM+HCl+EDC recycled from downstream condensation step) in contact with them. After quenching, the temperature of the gases is generally below 200° C., preferably below 180° C. and even more preferably, below 150° C. Such a low thermal content would not allow ensuring the thermal duty of the VCM column but it is sufficient to heat up the entry (feed) of said column according to the present invention.

At the end of step 2, when said step also comprises partial condensing, the temperature of the gases is generally comprised between 25 and 50° C. and the pressure is adapted between the pressure of step 1 and the operating pressure of the first separation step 3.

In a preferred embodiment of the invention, at least 2 condensers are used having each an inlet and an outlet stream and the heat exchanger is powered by at least part of the inlet stream of the last condenser.

Preferably, the first separation step 3 of the process according to the invention involves a distillation column that separates HCl on top from VCM and EDC at the bottom. This column is preferably operated under a pressure of from 9 to 14 barg. The HCl separated on top can be used in an oxychlorination unit (for instance for making EDC from ethylene) or for any other purpose. A refrigeration unit is preferably used on top of this column to liquefy the HCl required for the reflux of the column. Sieve trays or valves trays can be used in this column.

Preferably, the second separation step 4 of the process according to the invention involves a distillation column that separates VCM on top while unconverted EDC is purged at the bottom. This column is preferably operated under a pressure of from 4 to 8 barg depending on the temperature of the cooling fluid (usually cooling water) available for the condensation on top of the column. VCM required for the reflux of the column and produced VCM are condensed. Sieve trays or valves trays can be used in this column.

According to the invention, the heat exchanger may be of any type. It preferably is a multi-tubular heat exchanger, a spiral heat exchanger or a Compabloc® heat exchanger. Multi-tubular heat exchangers are more particularly preferred.

The present invention also relates to a process for the manufacture of PVC. To this effect, the invention relates to a process for the manufacture of PVC by polymerization of the VCM obtained by a process as described above.

The process for the manufacture of PVC may be a mass, solution or aqueous dispersion polymerization process; preferably, it is an aqueous dispersion polymerization process.

The expression “aqueous dispersion polymerization” is understood to mean free radical polymerization in aqueous suspension as well as free radical polymerization in aqueous emulsion and polymerization in aqueous microsuspension.

The expression “free radical polymerization in aqueous suspension” is understood to mean any free radical polymerization process performed in aqueous medium in the presence of dispersing agents and oil-soluble free radical initiators.

The expression “free radical polymerization in aqueous emulsion” is understood to mean any free radical polymerization process performed in aqueous medium in the presence of emulsifying agents and water-soluble free radical initiators.

The expression “aqueous microsuspension polymerization”, also called polymerization in homogenized aqueous dispersion, is understood to mean any free radical polymerization process in which oil-soluble initiators are used and an emulsion of droplets of monomers is prepared by virtue of a powerful mechanical stirring and the presence of emulsifying agents.

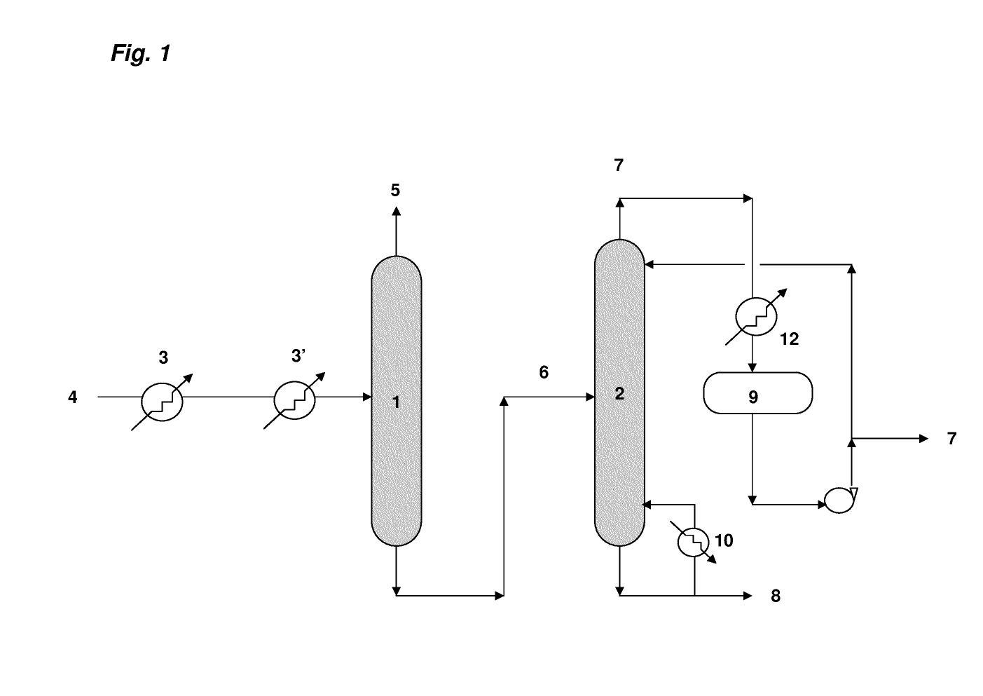

The present invention is illustrated in a non limitative way by FIGS. 1 to 3 attached, which show some preferred embodiments thereof. In these figures, identical reference numbers designate identical or similar items.

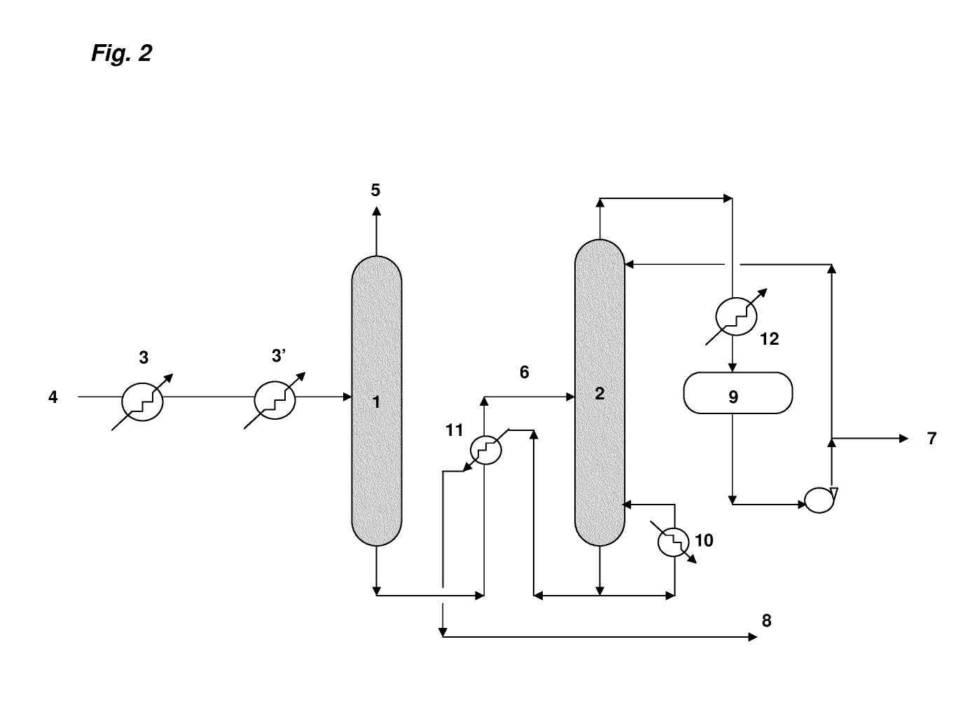

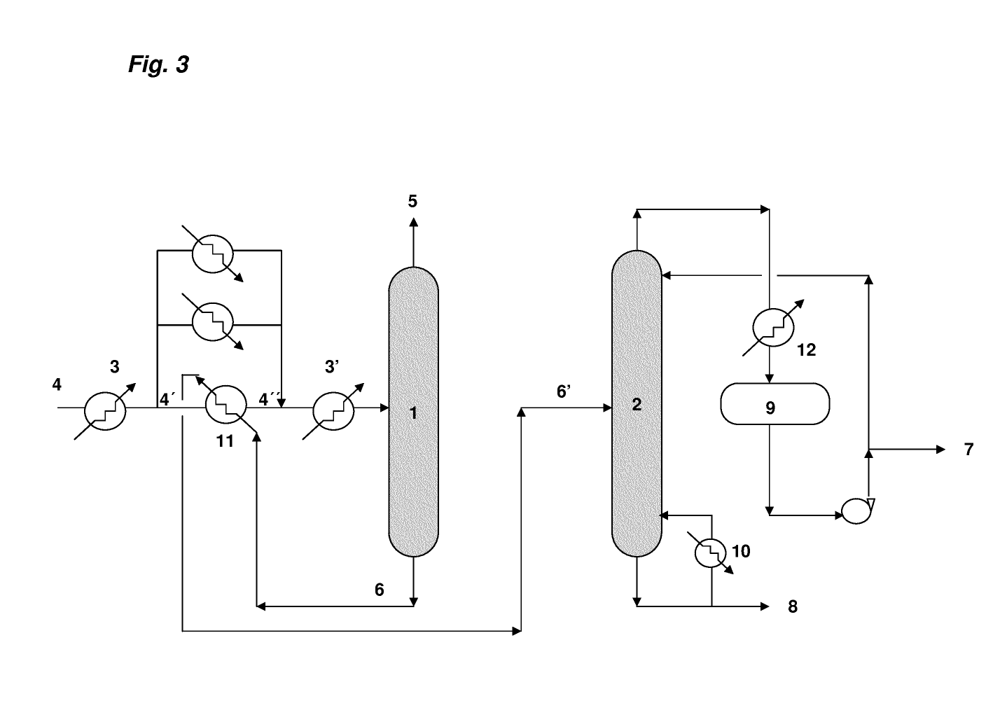

FIG. 1 shows a typical arrangement of HCl and VCM columns according to prior art, and FIGS. 2 and 3 show two different embodiments of arrangements according to the invention.

As can be seen from FIG. 1, a gaseous mixture (4) of (HCl+VCM+EDC) coming from an EDC pyrolysis section and its downstream quench unit (not shown) is first condensed in 2 condensers (3 and 3′), then separated in 2 steps:

HCl (5) is separated on top of the HCl column (1), and a mixture of (VCM+EDC) (6) is directed to the VCM column (2);

VCM (7) is separated on top of the VCM column (2);

unconverted EDC (8) is separated at the bottom of the VCM column and recycled to an EDC purification section (not shown).

In this typical arrangement, the (VCM+EDC) mixture (6) is directly sent to the VCM column (2), which has a VCM condenser (12) and a reflux drum (9) on the VCM stream, and a reboiler (10) at the bottom.

In a first embodiment of the invention, illustrated in FIG. 2, a heat exchanger (11) is installed between the feed (6) and the bottom of the VCM column (2). This arrangement leads to a reduction of the energy consumption of the reboiler (10), with a very low impact on the heat duty of the VCM condenser (12).

In a second embodiment of the invention, illustrated in FIG. 3, a heat exchanger (11) is installed between the feed (6) of the VCM column (2) and the HCl/VCM/EDC mixture coming from the pyrolysis (4) and its downstream quench unit (not shown), right before said mixture enters the second condenser (3′). As can be seen on this figure, not all the mixture coming from the pyrolysis passes through this heat exchanger (11) but instead, some of is by-passed. This arrangement also leads to a reduction of the energy consumption of the reboiler (10).

The embodiments described above have been the object of numerical simulations using ASPEN software, of which you will find the results in tables 1 and 2 below.

Table 1 is the result of a numerical simulation using version V7.2 of the Aspen software and comparing the classical layout (represented in FIG. 1) and the layout of FIG. 2 using the conditions set forth in said Table 1.

Table 2 is the result of a numerical simulation using version 2004.1 of the Aspen software and comparing the classical layout (represented in FIG. 1) and the layout of FIG. 3 using the conditions set forth in said Table 2.

As can be seen on these Tables, both the layout of FIG. 2 and the one of FIG. 3 lead to a substantial reduction of the duty (energy consumption) of the reboiler (10).

TABLE 1

| FIG. 1. | ID | 4 | 5 | 6 | 7 | 8 | 10 | 12 | | | |
|---|---|---|---|---|---|---|---|---|---|---|---|
| Mass flow rate | kg/s | 27.218 | 6.316 | 20.902 | 10.656 | 10.246 | x | x | | | |
| Volum. flow rate | m3/h | 892.389 | 974.749 | 82.366 | 43.166 | 36.178 | x | x | | | |
| Vapour fraction | kg/kg | 0.19 | 1.00 | 0.00 | 0.00 | 0.00 | x | x | | | |
| Temperature | C. | 34.6 | −27.7 | 89.6 | 31.7 | 147.6 | x | x | | | |
| Pressure | bar_a | 12.3 | 11.5 | 12.2 | 4.8 | 5.2 | x | x | | | |
| Duty | kW | x | x | x | x | x | 5557.92 | 5821.49 | | | |
| FIG. 2. | ID | 4 | 5 | 6 | 6′ | 7 | 8 | 8′ | 10 | 11 | 12 |
| Mass flow rate | kg/s | 27.22 | 6.32 | 20.90 | 20.90 | 10.66 | 10.25 | 10.25 | x | x | x |
| Volum. flow rate | m3/h | 892.39 | 974.75 | 1307.14 | 1976.58 | 43.17 | 36.18 | 32.44 | x | x | x |
| Vapour fraction | kg/kg | 0.19 | 1.00 | 0.22 | 0.32 | 0.00 | 0.00 | 0.00 | x | x | x |
| Temperature | C. | 34.6 | −27.7 | 58.3 | 62.6 | 31.7 | 147.6 | 77.6 | x | x | x |
| Pressure | bar_a | 12.3 | 11.5 | 5.2 | 5.0 | 4.8 | 5.2 | 4.8 | x | x | x |
| Duty | kW | x | x | x | x | x | x | x | 4557.1 | 1012.5 | 5833.3 |

TABLE 2

| FIG. 1. | ID | 4 | 5 | 6 | 7 | 8 | 10 | 12 | | | | |
|---|---|---|---|---|---|---|---|---|---|---|---|---|
| Mass flow rate | kg/s | 36.57 | 7.85 | 28.98 | 13.23 | 15.75 | x | x | | | | |
| Volum. flow rate | m3/h | 4747.9 | 1212.4 | 112.6 | 53.8 | 55.3 | x | x | | | | |
| Vapour fraction | kg/kg | 1 | 1 | 0 | 0 | 0 | x | x | | | | |
| Temperature | C. | 133.3 | −27.7 | 93.5 | 36.7 | 155.6 | x | x | | | | |
| Pressure | bar_a | 13.5 | 11.5 | 12.2 | 5.5 | 6.1 | x | x | | | | |
| Duty | kW | x | x | x | x | x | 8079 | 7820 | | | | |
| FIG. 3. | ID | 4 | 4′ | 4″ | 5 | 6 | 6′ | 7 | 8 | 10 | 11 | 12 |
| Mass flow rate | kg/s | 36.57 | 8.48 | 8.48 | 7.85 | 28.99 | 28.99 | 13.23 | 15.75 | x | x | x |
| Volum. flow rate | m3/h | 4748.1 | 1101.6 | 791.0 | 1212.4 | 112.7 | 483.2 | 53.8 | 55.3 | x | x | x |
| Vapour fraction | kg/kg | 1.00 | 1.00 | 0.65 | 1.00 | 0.00 | 0.11 | 0.00 | 0.00 | x | x | x |
| Temperature | C. | 133.3 | 133.3 | 103.5 | -27.7 | 93.5 | 97.3 | 36.7 | 155.6 | x | x | x |
| Pressure | bar_a | 13.5 | 13.5 | 13.3 | 11.5 | 12.2 | 12.0 | 5.5 | 6.1 | x | x | x |
| Duty | kW | x | x | x | x | x | x | x | x | 6979 | 1100 | 7820 |

1- A process for manufacturing vinyl chloride monomer (VCM), comprising:
step 1). subjecting 1,2-dichloroethane (EDC) to pyrolysis in order to generate a gas mixture comprising VCM, hydrochloric acid (HCl), and EDC;
step 2). quenching said gas mixture and eventually further cooling, or condensing, or both said gas mixture to form a liquid-and-gas mixture;
step 3). subjecting said liquid-and-gas mixture to a first separation step to remove substantially all the HCl from said liquid-and-gas mixture so as to leave a stream consisting substantially of VCM and EDC; and
step 4). subjecting said stream consisting substantially of VCM and EDC to a second separation step so as to get a stream of substantially pure VCM and a stream of unconverted EDC,
wherein a heat exchanger is used to heat up said stream consisting substantially of VCM and EDC prior to being fed to a distillation column in step 4), said heat exchanger being powered by a stream of hot fluid available in any one step of the process selected from the group consisting of steps 2), step 3), and step 4) but said stream of hot fluid being available after the quenching performed in step 2).

2- The process according to claim 1, wherein the heat exchanger is powered by at least part of said stream of unconverted EDC obtained in step 4).

3- The process according to claim 1, wherein the heat exchanger is powered by a stream of hot mixture comprising VCM, HCl and EDC available in step 2) after said quenching.

4- The process according to claim 1, wherein in step 2), said gas mixture leaving step 1) is first cooled down in a quench device and thereafter, partially condensed using at least one condenser.

5- The process according to claim 4, wherein the quench device uses a liquid quench medium which is a liquid mixture of VCM, HCl, and EDC, said liquid mixture being recycled from a downstream condensation step.

6- The process according to claim 4 or 5, wherein at least 2 successive condensers having each an inlet stream and an outlet stream are used in step 2), one of said condensers being a last condenser, and wherein the heat exchanger is powered by at least part of the inlet stream of the last condenser.

7- The process according to claim 1, wherein the first separation step 3) involves a distillation column with a top and a bottom that separates HCl on top from VCM and EDC at the bottom.

8- The process according to claim 1, wherein in the second separation step 4) said distillation column being fed said stream consisting substantially of VCM and EDC has a top and a bottom and separates VCM on said top while unconverted EDC is purged at said bottom.

9- The process according to claim 1, wherein the heat exchanger is a multi-tubular heat exchanger, a spiral heat exchanger or a Compabloc® heat exchanger.

10- The process according to claim 9, wherein the heat exchanger is a multi-tubular heat exchanger.

11- A process for the manufacture of polyvinyl chloride (PVC) by polymerization of the vinyl chloride monomer obtained by the process according to claim 1.

12- The process according to claim 1, wherein in the second separation step 4), said distillation column being fed said stream consisting substantially of VCM and EDC has a reboiler, and wherein said process provides a reduction in energy consumption of said reboiler compared to a process for manufacturing VCM comprising said steps 1) to 4) without using said heat exchanger.
